(19) United States
(12) Patent Application Publication
(10) Pub. No.: US 2022/0110581 A1
(43) Pub. Date: Apr. 14, 2022

(54) MONITORING SKIN HEALTH

(71) Applicant: X Development LLC, Mountain View, CA (US)

(72) Inventor: Anupama Thubagere Jagadeesh, San Jose, CA (US)

(21) Appl. No.: 17/067,402

(22) Filed: Oct. 9, 2020

(51) Int. Cl. A61B 5/00; G06N 3/08

(52) U.S. Cl. CPC A61B 5/442; A61B 5/742; G06N 3/08; A61B 5/7275; A61B 5/444

(57) ABSTRACT

Methods, systems, and apparatus, including computer programs encoded on a computer storage medium for skin health monitoring including instructions that, when executed, cause the one or more processors to perform various operations. The operations include obtaining first scan data representing a first hyperspectral scan of a user's skin at a first time. The operations include obtaining second scan data representing one or more previous hyperspectral scans of the user's skin during a period of time prior the first time. The operations include determining, based on providing the first scan data and the second scan data as input features to a machine learning model, a likelihood that the user will develop a predicted skin condition in the future. The operations include providing, for display on a user computing device associated with the user, information about the predicted skin condition.

### TECHNICAL FIELD

This disclosure generally relates to monitoring and predicting skin health. For instance, implementations of the disclosure relate to using machine learning to anticipate an individual's future skin condition based on previously measured skin conditions of the individual.

### SUMMARY

In general, the disclosure relates to a machine learning system that predicts future changes in individual skin health based on environmental factors and the current condition of the individual's skin. The system can provide a probabilistic output indicating potential future skin conditions, and a recommended optimal solution for maintaining skin health.

In general, innovative aspects of the subject matter described in this specification can be embodied in methods that include the actions of obtaining first scan data representing a first hyperspectral scan of a user's skin at a first time. The actions include obtaining second scan data representing one or more previous hyperspectral scans of the user's skin during a period of time prior the first time. The actions include determining, based on providing the first scan data and the second scan data as input features to a machine learning model, a likelihood that the user will develop a predicted skin condition in the future. The actions include providing, for display on a user computing device associated with the user, information about the predicted skin condition. Other implementations of this aspect include corresponding systems, apparatus, and computer programs, configured to perform the actions of the methods, encoded on computer storage devices. These and other implementations can each optionally include one or more of the following features.

In some implementations, the machine learning model includes a neural network. In some implementations, the predicted skin condition includes at least one of acne, wrinkles, pores, discolorations, hyperpigmentation, spots, blackheads, whiteheads, dry patches, moles, or psoriasis.

In some implementations, determining the likelihood that the user will develop the predicted skin condition in the future includes identifying a change in a region of the user's skin, and identifying the predicted skin condition based on determining that the change correlates to a symptom of the predicted skin condition. In some implementations, the change in the region of the user's skin includes a change in moisture content. In some implementations, the change in the region of the user's skin includes a change in coloration.

Some implementations include obtaining data indicating environmental conditions associated with the user. In some implementations, determining the likelihood that the user will develop the predicted skin condition in the future includes determining the likelihood that the user will develop the predicted skin condition in the future based on providing the first scan data, the second scan data, and the data indicating environmental conditions associated with the user as input features to a machine learning model.

Some implementations include obtaining medical information associated with the user. In some implementations, determining the likelihood that the user will develop the predicted skin condition in the future includes determining the likelihood that the user will develop the predicted skin condition in the future based on providing the first scan data, the second scan data, and the medical information associated with the user as input features to a machine learning model.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1A depicts an exemplary device for recording hyperspectral scans of a user's skin.

FIG. 1B depicts a block diagram of the device of FIG. 1.

FIG. 2 depicts a block diagram of an exemplary machine learning system for predicting skin health.

FIG. 3 is a flowchart illustrating an exemplary method for monitoring skin health.

FIG. 4 depicts a schematic diagram of a computer system that may be applied to any of the computer-implemented methods and other techniques described herein.

Like reference numbers and designations in the various drawings indicate like elements.

### DETAILED DESCRIPTION

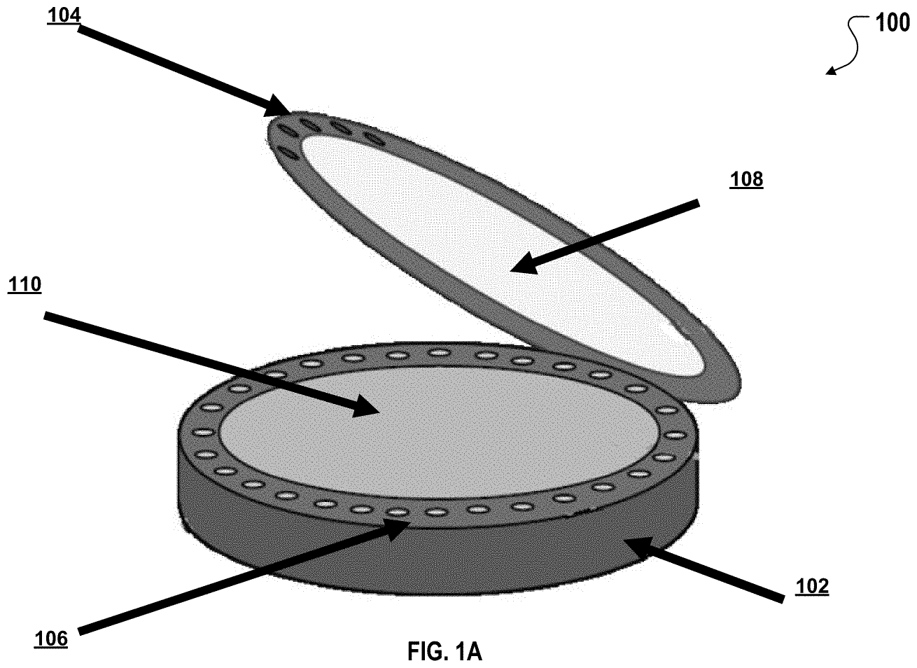

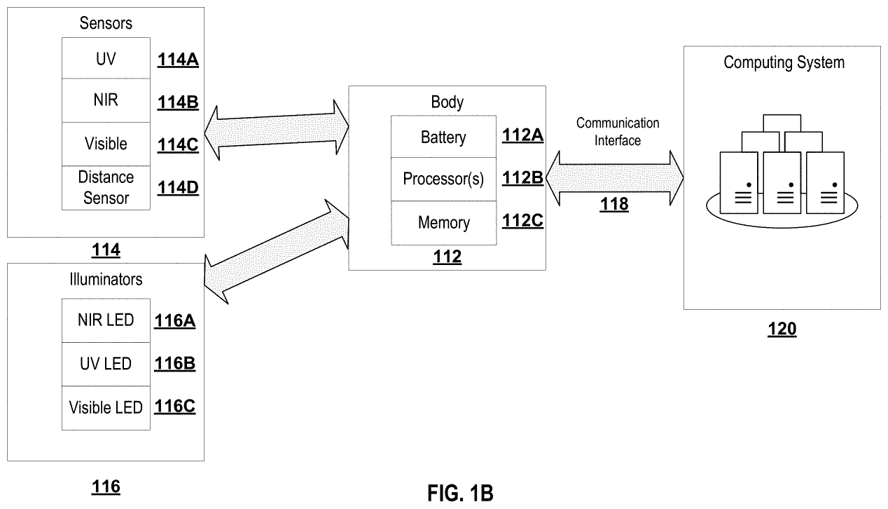

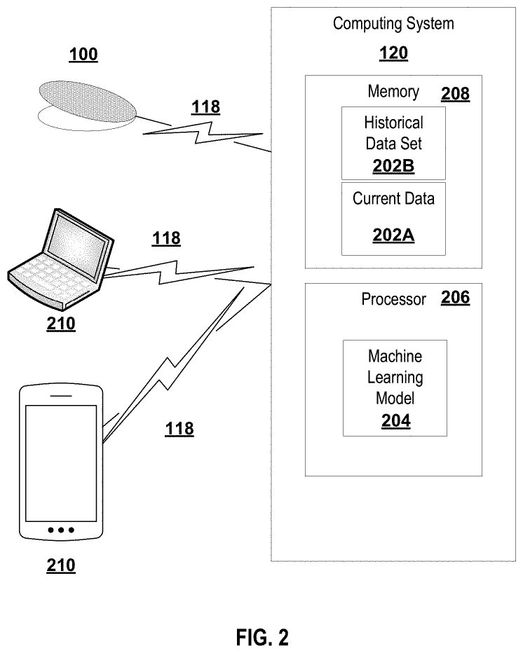

FIG. 1A depicts an exemplary device for recording hyperspectral scans of a user's skin. In the illustrated example, the device is implemented as a makeup compact device, however, the device 100 can be implemented in other shapes and structures (e.g., a mirror, cell phone, etc.). For example, the device 100 can include a body 102, sensors 104, illuminators 106, one or more processors (element 112B of FIG. 2), and a communication interface. The sensors 104 can be image sensors, configured to receive light that is emitted from the illuminators 106 and reflected off a user's skin. The image sensors can receive light at specific wavelengths, such as near-IR (NIR), visible, and ultra violet (UV) light or any combination thereof. For example, the sensors 104 can detect light between 300 nm and 1000 nm wavelengths. In some examples, the image sensors can be wavelength controlled. For example, wavelength controlled image sensors are able to detect specific light polarizations (e.g. parallel, cross or circular polarized light). The image sensors are capable of collecting a hyperspectral scan using standard photography, fluorescent photography, polarized photography and video. The image sensors can be, but are not limited to, any suitable device such as a camera that includes charge-coupled device (CCD) or a complementary metal oxide-semiconductor (CMOS) sensor.

The illuminators 106 can be arranged to emit light corresponding to the range of wavelengths the sensors 104 detect. The illuminators 106 can be but are not limited to light emitting diodes (LEDs).

In some implementations, the device houses a mirror 108 and powder 110. The powder 110 can be any chemical compound necessary for maintenance or for cosmetic or pathogenic intervention. The body 102 can also include a battery, processor and the electronics required for operating the illumination array 106 and the sensor array 104. In some instances the device 100 may also include a distance sensor (not shown).

In some implementations, the device is a makeup compact, having a clamshell form. The sensors 104 can be in the upper clamshell, and the illuminators 106 can be on the lower clamshell, the upper clamshell, or both. In this implementation a hyperspectral scan of the user's face can be captured while the user moves the makeup compact to view their face in a natural manner. In other words, the skin monitoring device can capture images of the user's face from various perspectives without requiring the user to follow a regimented series of motions because, for example, the natural movements that the user performs with the device as a makeup compact will permit the image sensors to capture the user's face from different perspectives (e.g., angles of view). Alternatively, the device 100 can provide the user instructions to perform a series of movements in order to obtain a comprehensive scan of the user's face.

In some implementations, the device 100 is a mobile device (e.g. smartphone, tablet, laptop, etc.) The sensors 104 and illuminators 106 can be mounted to the face of the mobile device, or the rear. In this implementation the device 100 can record a hyperspectral scan during routine use, or can prompt the user to position the device 100 in a series of locations, to enable a detailed scan of an area of the user's skin (e.g. face). In some implementations, the device 100 is a mirror (e.g. handheld vanity mirror, bathroom wall mirror etc.).

FIG. 1B depicts an example schematic diagram of device 100. The device can have an array of sensors 114. The sensors can be image sensors, including one or more of UV sensors 114A, NIR sensors 114B, visible sensors 114C, and distance sensors 114D. The device can also include an array of illuminators 116, which can contain one or more NIR LEDs 116A, UV LEDs 116B, and Visible LEDs 116C. These arrays can optionally be housed within the body 112, or can be separate from the body 112. A processor or multiple processors 112B can be housed within the body, and can handle the activation, recording and operation of both the sensors 114 and the illuminators 116, as well as the memory 112C. The memory can be a non-transitory memory used for storing data temporarily before it can be transmitted to the computing system 120 via the communications interface 118. In some instances one or more batteries 112A can provide electrical power to the device 100.

The communication interface 118 provides communications for the device 100 with the computing system 120. The communication interface 118 can be but is not limited to a wired communication interface (e.g., USB, Ethernet, fiber optic) or wireless communication interface (e.g., Bluetooth, ZigBee, WiFi, infrared (IR), CDMA2000, etc). The communication interface 118 can be used to communicate directly or indirectly, e.g., through a network, with the computing system 120.

The device 100 is configured to collect hyperspectral scans of a user's skin and transmit them to a computing system 120. Hyperspectral scans can be triggered by a user input, or automatically using a proximity sensor, or by any other suitable means. A hyperspectral scan can be a scan of the light reflected from a user's skin, over a broad range of the light spectrum. For example 300 nm-1000 nm wavelengths. The hyperspectral scan can include a plurality of images that can be stitched together to form a two-dimensional or three-dimensional representation of a portion of a user's skin, for example, their face. The hyperspectral scan can provide a detailed map of a user's skin condition, showing a broad range of blemishes or imperfections.

FIG. 2 depicts an implementation of a computing system 120 which incorporates a machine learning model 204 to identify patterns in hyperspectral scans that are predictive of future skin conditions which may require care. In some implementations, computing system 120 is a cloud-based computing system, or a remote computing platform. For example, the computing system 120 can include a machine learning model 204 that has been trained to receive model inputs, e.g., skin factors, present skin condition, medical information, and environmental factors, and to generate a predicted output, e.g., a prediction of the likelihood of an acne breakout, rash, dry irritated skin, etc. For example, the computing system 120 can detect a localized area of discoloration within a hyperspectral scan of a user's face. Based on the size, location, and shape of the discoloration, as well as the user's historical scans, and present environmental parameters, the computing system 120 can determine that there is a high likelihood of the user developing a blemish or other skin condition in the localized area of discoloration.

In some implementations, the machine learning model 204 (or portions thereof) can be executed by the skin monitoring device 100. For example, operations of the machine learning model 204 can be executed by the skin monitoring device 100. In some examples, operations of the machine learning model 204 can be distributed between the skin monitoring device 100 and the computing system 120.

The computing system 120 receives present skin data 202A from the device 100 via the communications interface 118. The computing system 120 can also receive present skin data 202A from other user devices 210, or a network. In some implementations the present skin data can be received in real-time. The present skin data 202A is then used by the machine learning model 204 to generate the predicted output. The present skin data 202A can include one of, or any combination of skin factors, present skin condition, medical information, and environmental factors, among other things.

Skin factors can include, but are not limited to, a user's pigmentation, wrinkles, texture, pores, reflectance, pigmentation, and discoloration. Skin factors may be obtained through user input, or developed over time from previous hyperspectral scans that have been stored in a repository.

Present skin conditions can include, but are not limited to, blemishes (e.g. acne, whiteheads, blackheads, etc.), discoloration (e.g. sunburn, rash, etc.), amino acid content (e.g. tryptophan, peptides, tyrosine etc.), dryness and moisture content, concentration of chemical compounds (e.g. coproporphyrin, nicotinamide adenine dinucleotide (NADH), triglycerides, lipids, fatty acids, etc.), or other skin imperfections. Present skin conditions can be obtained from recent hyperspectral scans, or input from a user, the present solution is not limited thereto.

Medical information can include, but are not limited to, a user's age, activity levels, diet, current or past medications, and any other pertinent medical history. The user may volunteer this information, for example, during an account registration step, or when prompted by the computing system 120 via a communications interface 118.

Environmental factors can include, but are not limited to, the temperature, relative humidity, pollen count, UV index, etc. Environmental factors may be obtained via a known user's location, or by additional sensors on the device 100, among other things.

The computing system 120 can store in memory a historical data set 202B for a user. The historical data set can include all data that has previously been used, or a subset of the previous data. The historical data set 202B can also include data relating to common trends seen across multiple individuals, among other things.

The machine learning model 204 receives the present skin data 202A, and the historical data 202B and generates a predictive output. For example, the machine learning model 204 can compare the present skin data (e.g., present hyperspectral scan images of the user's skin) with historical data (e.g., historical hyperspectral can images of the user's skin) to identify changes in the user's skin health. For example, the machine learning model 204 can identify, and in some implementations locate, minute changes in the regions of the user's skin, such as changes of moisture content, changes in coloration, hyperpigmentation, blood flow, or a combination thereof. The machine learning model 204 can correlate the detected changes in the user's skin with known patterns of skin health (e.g., a library of skin symptoms that lead to various skin conditions) to generate a predictive output of the user's future skin health. The predictive output can include, but is not limited to a type of future skin condition that the user is likely to experience, a location of a predicted skin condition, or a combination thereof.

In some implementations, the machine learning model 204 incorporates additional data such as environmental factors associated with the user, the user's medical information, or a combination thereof, in order to generate a predictive output. For example, the machine learning model 204 can correlate the identified changes in the user's skin, with the environmental conditions the user is subject to and/or the user's medical information to identify predicted skin conditions that the user is likely to experience.

In some implementations, the machine learning model 204 is a deep learning model that employs multiple layers of models to generate an output for a received input. A deep neural network is a deep machine learning model that includes an output layer and one or more hidden layers that each apply a non-linear transformation to a received input to generate an output. In some cases, the neural network may be a recurrent neural network. A recurrent neural network is a neural network that receives an input sequence and generates an output sequence from the input sequence. In particular, a recurrent neural network uses some or all of the internal state of the network after processing a previous input in the input sequence to generate an output from the current input in the input sequence. In some other implementations, the machine learning model 204 is a convolutional neural network. In some implementations, the machine learning model 204 is an ensemble of models that may include all or a subset of the architectures described above.

In some implementations, the machine learning model 204 can be a feedforward autoencoder neural network. For example, the machine learning model 204 can be a three-layer autoencoder neural network. The machine learning model 204 may include an input layer, a hidden layer, and an output layer. In some implementations, the neural network has no recurrent connections between layers. Each layer of the neural network may be fully connected to the next, e.g., there may be no pruning between the layers. The neural network may include an optimizer for training the network and computing updated layer weights, such as, but not limited to, ADAM, Adagrad, Adadelta, RMSprop, Stochastic Gradient Descent (SGD), or SGD with momentum. In some implementations, the neural network may apply a mathematical transformation, e.g., a convolutional transformation or factor analysis to input data prior to feeding the input data to the network.

In some implementations, the machine learning model 204 can be a supervised model. For example, for each input provided to the model during training, the machine learning model 204 can be instructed as to what the correct output should be. The machine learning model 204 can use batch training, e.g., training on a subset of examples before each adjustment, instead of the entire available set of examples. This may improve the efficiency of training the model and may improve the generalizability of the model. The machine learning model 204 may use folded cross-validation. For example, some fraction (the “fold”) of the data available for training can be left out of training and used in a later testing phase to confirm how well the model generalizes. In some implementations, the machine learning model 204 may be an unsupervised model. For example, the model may adjust itself based on mathematical distances between examples rather than based on feedback on its performance.

A machine learning model 204 can be trained to recognize patterns in skin condition when compared with the historical data of an individual, and environmental parameters. In some examples, the machine learning model 204 can be trained on hundreds of hyperspectral scans of an individual's skin. The machine learning model 204 can be trained to identify potential breakouts and signs of future skin care needs.

The machine learning model 204 can be, for example, a deep-learning neural network or a “very” deep learning neural network. For example, the machine learning model 204 can be a convolutional neural network. The machine learning model 204 can be a recurrent network. The machine learning model 204 can have residual connections or dense connections. The machine learning model 204 can be an ensemble of all or a subset of these architectures. The machine learning model 204 is trained to predict the likelihood that a user will experience a skin condition requiring care within a period of time in the future based on detecting patterns indicative of future skin conditions from one or more of the present skin data 202A and the historical data set 202B. The model may be trained in a supervised or unsupervised manner. In some examples, the model may be trained in an adversarial manner. In some examples, the model may be trained using multiple objectives, loss functions or tasks.

The machine learning model 204 can be configured to provide a binary output, e.g., a yes or no indication of whether the user's skin is in a healthy condition. In some examples, the machine learning model 204 is configured to determine a type of the predicted skin condition. For example, based on the present and historical data, the machine learning model can determine that the user is likely to experience a particular type of skin condition in the future. Types of skin conditions that can be detected include, but are not limited to, acne, wrinkles, pores, discolorations, hyperpigmentation, spots, blackheads, whiteheads, dry patches, moles, and psoriasis. In some implementations, the output data of the machine learning model 204 can be used for orthogonal diagnosis of women's health (e.g., ovulation).

In some implementations, the machine learning model 204 can provide suggested treatment options for the user to treat the predicted skin condition. For example, the computing system 120 can send the predictive output data to the user's dermatologist. Specifically, the computing system 120 can send the predictive output data to a computing device registered to the user's dermatologist. In some implementations, the computing system 120 can provide recommendations for a skincare product that treats or helps to prevent the predicted skin condition. Specifically, the computing system 120 can send the recommendations to a computing device 210 associated with the user.

Further to the descriptions above, a user may be provided with controls allowing the user to make an election as to both if and when systems, programs, or features described herein may enable collection of user information. In addition, certain data may be treated in one or more ways before it is stored or used, so that personally identifiable information is removed. For example, a user's identity may be treated so that no personally identifiable information can be determined for the user, or a user's test data and/or diagnosis cannot be identified as being associated with the user. Thus, the user may have control over what information is collected about the user and how that information is used.

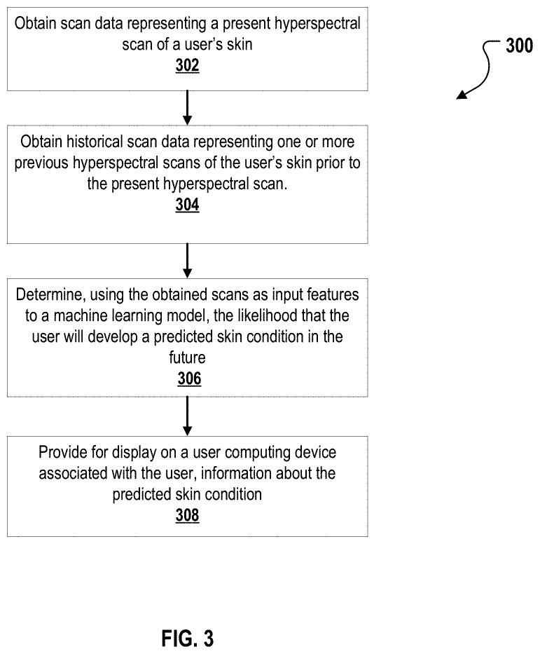

FIG. 3 is a flowchart illustrating an exemplary method for monitoring skin health. For clarity of presentation, the description that follows generally describes method 300 in the context of the other figures in this description. However, it will be understood that method 300 can be performed, for example, by any system, environment, software, and hardware, or a combination of systems, environments, software, and hardware, as appropriate. In some implementations, various steps of method 300 can be run in parallel, in combination, in loops, or in any order.

The computing system obtains a first scan data that represents a hyperspectral scan of a user's skin at a first time (302). For example, a hyperspectral scan can be received via the communications interface 118, from the device 100, when a user completes a scan of their skin. The hyperspectral scan data can include any suitable combination of skin factors, present skin condition, medical information, and environmental factors, among other things as described above. The hyperspectral scan can cover, but is not limited to, a 300 nm-1000 nm wavelength of light.

The computing system obtains second scan data that represents one or more previous hyperspectral scans of the user's skin during a time period prior to the first time (304). This data can be referred to as historical data, and can include additional information that was not provided at the time of the previous hyperspectral scans.

A determination is made based on providing the first scan data and the second scan data to a machine learning model (306). The machine learning model can be as described above. The machine learning model will determine the likelihood that the user will develop a predicted skin condition (e.g. as rash, dry skin, acne, etc.) in the future.

Information about the predicted skin condition is provided, by the one or more processors for display on a user computing device associated with the user (308). The user computing device may be a mobile device (e.g. cell phone, table, PDA, etc.) or a personal computer (e.g. desktop, laptop, etc.) among other things.

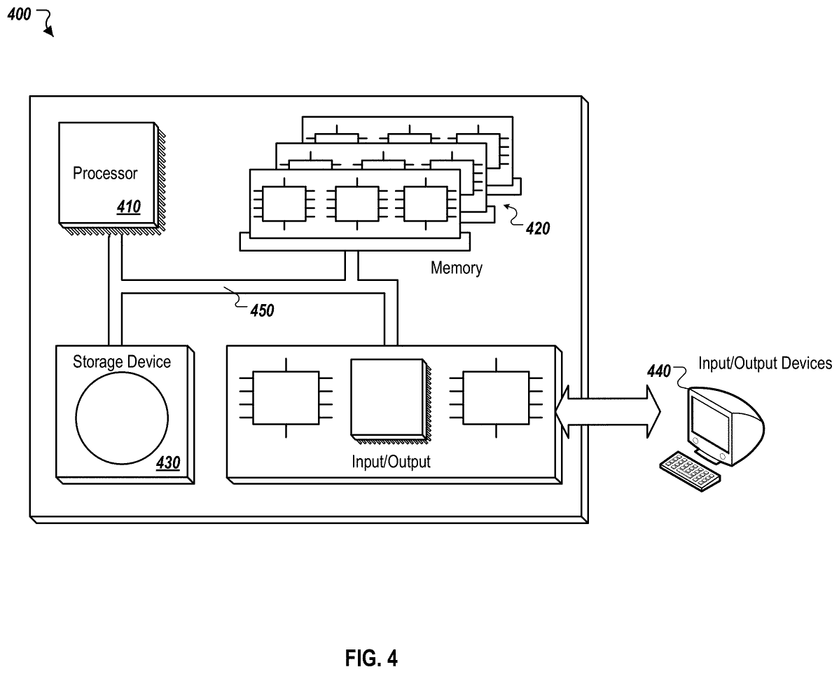

FIG. 4 is an example of a computing system. The system 400 can be used to carry out the operations described in association with any of the computer-implemented methods described previously, according to some implementations. In some implementations, computing systems, devices and the functional operations described in this specification can be implemented in digital electronic circuitry, in tangibly-embodied computer software or firmware, in computer hardware, including the structures disclosed in this specification (e.g., system 400) and their structural equivalents, or in combinations of one or more of them. The system 400 is intended to include various forms of digital computers, such as laptops, desktops, workstations, personal digital assistants, servers, blade servers, mainframes, and other appropriate computers, including vehicles installed on base units or pod units of modular vehicles. The system 400 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 400 includes a processor 410, a memory 420, a storage device 430, and an input/output device 440. Each of the components 410, 420, 430, and 440 are interconnected using a system bus 450. The processor 410 is capable of processing instructions for execution within the system 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 may be a floppy disk device, a hard disk device, an optical disk device, or a tape device.

The input/output device 440 provides input/output operations for the system 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits). The machine learning model can run on Graphic Processing Units (GPUs) or custom machine learning inference accelerator hardware.

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

Embodiments and the operations described in this specification can be implemented in digital electronic circuitry, or in computer software, firmware, or hardware, including the structures disclosed in this specification or in combinations of one or more of them. The operations can be implemented as operations performed by a data processing apparatus on data stored on one or more computer-readable storage devices or received from other sources. A data processing apparatus, computer, or computing device may encompass apparatuses, devices, and machines for processing data, including by way of example a programmable processor, a computer, a system on a chip, or multiple ones, or combinations, of the foregoing. The apparatus can include special purpose logic circuitry, for example, a central processing unit (CPU), a field programmable gate array (FPGA) or an application-specific integrated circuit (ASIC). The apparatus can also include code that creates an execution environment for the computer program in question, for example, code that constitutes processor firmware, a protocol stack, a database management system, an operating system (for example an operating system or a combination of operating systems), a cross-platform runtime environment, a virtual machine, or a combination of one or more of them. The apparatus and execution environment can realize various different computing model infrastructures, such as web services, distributed computing and grid computing infrastructures.

A computer program (also known, for example, as a program, software, software application, software module, software unit, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A program can be stored in a portion of a file that holds other programs or data (for example, one or more scripts stored in a markup language document), in a single file dedicated to the program in question, or in multiple coordinated files (for example, files that store one or more modules, sub-programs, or portions of code). A computer program can be executed on one computer or on multiple computers that are located at one site or distributed across multiple sites and interconnected by a communication network.

Processors for execution of a computer program include, by way of example, both general- and special-purpose microprocessors, and any one or more processors of any kind of digital computer. Generally, a processor will receive instructions and data from a read-only memory or a random-access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data. A computer can be embedded in another device, for example, a mobile device, a personal digital assistant (PDA), a game console, a Global Positioning System (GPS) receiver, or a portable storage device. Devices suitable for storing computer program instructions and data include non-volatile memory, media and memory devices, including, by way of example, semiconductor memory devices, magnetic disks, and magneto-optical disks. The processor and the memory can be supplemented by, or incorporated in, special-purpose logic circuitry.

Mobile devices can include handsets, user equipment (UE), mobile telephones (for example, smartphones), tablets, wearable devices (for example, smart watches and smart eyeglasses), implanted devices within the human body (for example, biosensors, cochlear implants), or other types of mobile devices. The mobile devices can communicate wirelessly (for example, using radio frequency (RF) signals) to various communication networks (described below). The mobile devices can include sensors for determining characteristics of the mobile device's current environment. The sensors can include cameras, microphones, proximity sensors, GPS sensors, motion sensors, accelerometers, ambient light sensors, moisture sensors, gyroscopes, compasses, barometers, fingerprint sensors, facial recognition systems, RF sensors (for example, Wi-Fi and cellular radios), thermal sensors, or other types of sensors. For example, the cameras can include a forward- or rear-facing camera with movable or fixed lenses, a flash, an image sensor, and an image processor. The camera can be a megapixel camera capable of capturing details for facial and/or iris recognition. The camera along with a data processor and authentication information stored in memory or accessed remotely can form a facial recognition system. The facial recognition system or one-or-more sensors, for example, microphones, motion sensors, accelerometers, GPS sensors, or RF sensors, can be used for user authentication.

To provide for interaction with a user, embodiments can be implemented on a computer having a display device and an input device, for example, a liquid crystal display (LCD) or organic light-emitting diode (OLED)/virtual-reality (VR)/augmented-reality (AR) display for displaying information to the user and a touchscreen, keyboard, and a pointing device by which the user can provide input to the computer. Other kinds of devices can be used to provide for interaction with a user as well; for example, feedback provided to the user can be any form of sensory feedback, for example, visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments can be implemented using computing devices interconnected by any form or medium of wireline or wireless digital data communication (or combination thereof), for example, a communication network. Examples of interconnected devices are a client and a server generally remote from each other that typically interact through a communication network. A client, for example, a mobile device, can carry out transactions itself, with a server, or through a server, for example, performing buy, sell, pay, give, send, or loan transactions, or authorizing the same. Such transactions may be in real time such that an action and a response are temporally proximate; for example an individual perceives the action and the response occurring substantially simultaneously, the time difference for a response following the individual's action is less than 1 millisecond (ms) or less than 1 second (s), or the response is without intentional delay taking into account processing limitations of the system.

Examples of communication networks include a local area network (LAN), a radio access network (RAN), a metropolitan area network (MAN), and a wide area network (WAN). The communication network can include all or a portion of the Internet, another communication network, or a combination of communication networks. Information can be transmitted on the communication network according to various protocols and standards, including Long Term Evolution (LTE), 5G, IEEE 802, Internet Protocol (IP), or other protocols or combinations of protocols. The communication network can transmit voice, video, biometric, or authentication data, or other information between the connected computing devices.

Features described as separate implementations may be implemented, in combination, in a single implementation, while features described as a single implementation may be implemented in multiple implementations, separately, or in any suitable sub-combination. Operations described and claimed in a particular order should not be understood as requiring that the particular order, nor that all illustrated operations must be performed (some operations can be optional). As appropriate, multitasking or parallel-processing (or a combination of multitasking and parallel-processing) can be performed.

1. A computer-implemented skin health monitoring method executed by one or more processors, the method comprising:
obtaining, by the one or more processors, first scan data representing a first hyperspectral scan of a user's skin at a first time;
obtaining, by the one or more processors, second scan data representing one or more previous hyperspectral scans of the user's skin during a period of time prior the first time;
determining, based on providing the first scan data and the second scan data as input features to a machine learning model, a likelihood that the user will develop a predicted skin condition in the future; and
providing, by the one or more processors for display on a user computing device associated with the user, information about the predicted skin condition.

2. The method of claim 1, wherein the machine learning model comprises a neural network.

3. The method of claim 1, wherein the predicted skin condition comprise at least one of acne, wrinkles, pores, discolorations, hyperpigmentation, spots, blackheads, whiteheads, dry patches, moles, or psoriasis.

4. The method of claim 1, wherein determining the likelihood that the user will develop the predicted skin condition in the future comprises:
identifying a change in a region of the user's skin; and
identifying the predicted skin condition based on determining that the change correlates to a symptom of the predicted skin condition.

5. The method of claim 4, wherein the change in the region of the user's skin comprises a change in moisture content.

6. The method of claim 4, wherein the change in the region of the user's skin comprises a change in coloration.

7. The method of claim 1, further comprising obtaining data indicating environmental conditions associated with the user.

8. The method of claim 7, wherein determining the likelihood that the user will develop the predicted skin condition in the future comprises determining the likelihood that the user will develop the predicted skin condition in the future based on providing the first scan data, the second scan data, and the data indicating environmental conditions associated with the user as input features to a machine learning model.

9. The method of claim 1, further comprising obtaining medical information associated with the user.

10. The method of claim 9, wherein determining the likelihood that the user will develop the predicted skin condition in the future comprises determining the likelihood that the user will develop the predicted skin condition in the future based on providing the first scan data, the second scan data, and the medical information associated with the user as input features to a machine learning model.

11. One or more non-transitory computer readable storage media storing instructions that, when executed by at least one processor, cause the at least one processor to perform operations comprising:
obtaining, by the one or more processors, first scan data representing a first hyperspectral scan of a user's skin at a first time;
obtaining, by the one or more processors, second scan data representing one or more previous hyperspectral scans of the user's skin during a period of time prior the first time; determining, based on providing the first scan data and the second scan data as input features to a machine learning model, a likelihood that the user will develop a predicted skin condition in the future; and
providing, by the one or more processors for display on a user computing device associated with the user, information about the predicted skin condition.

12. The media of claim 11, wherein the machine learning model comprises a neural network.

13. The media of claim 11, wherein the predicted skin condition comprise at least one of acne, wrinkles, pores, discolorations, hyperpigmentation, spots, blackheads, whiteheads, dry patches, moles, or psoriasis.

14. The media of claim 11, wherein determining the likelihood that the user will develop the predicted skin condition in the future comprises:
identifying a change in a region of the user's skin; and
identifying the predicted skin condition based on determining that the change correlates to a symptom of the predicted skin condition.

15. The media of claim 14, wherein the change in the region of the user's skin comprises a change in moisture content.

16. The media of claim 14, wherein the change in the region of the user's skin comprises a change in coloration.

17. The media of claim 11, further comprising obtaining data indicating environmental conditions associated with the user.

18. The media of claim 17, wherein determining the likelihood that the user will develop the predicted skin condition in the future comprises determining the likelihood that the user will develop the predicted skin condition in the future based on providing the first scan data, the second scan data, and the data indicating environmental conditions associated with the user as input features to a machine learning model.

19. The media of claim 11, further comprising obtaining medical information associated with the user.

20. The media of claim 19, wherein determining the likelihood that the user will develop the predicted skin condition in the future comprises determining the likelihood that the user will develop the predicted skin condition in the future based on providing the first scan data, the second scan data, and the medical information associated with the user as input features to a machine learning model.
